(19) United States
(12) Patent Application Publication
(10) Pub. No.: US 2022/0220491 A1
(43) Pub. Date: Jul. 14, 2022

(54) A METHOD FOR RECOMBINANT PROTEIN PRODUCTION

(71) Applicant: ATATÜRK ÜNIVERSITESI BILIMSEL ARASTIRMA PROJELERI BIRIMI, Erzurum (TR)

(72) Inventors: Mesut TASKIN, Erzurum (TR); Yagmur ÜNVER, Erzurum (TR); Melike YILDIZ, Erzurum (TR); Melek ACAR, Erzurum (TR); Seyda YILDIZ, Erzurum (TR)

(21) Appl. No.: 17/607,185

(22) PCT Filed: Dec. 16, 2020
(86) PCT No.: PCT/TR2020/051309

§ 371 Date: Oct. 28, 2021

(30) Foreign Application Priority Data
Dec. 19, 2019 (TR) 2019/20838

(51) Int. Cl. C12N 15/81; C12N 9/04; C07K 14/435; C07K 14/47

(52) U.S. Cl. CPC C12N 15/815; C12N 9/0006; C12N 2830/002; C07K 14/43572; C07K 14/4703; C12Y 101/03013

(57) ABSTRACT

A method including the process steps of insertion of a gene to be expressed into the plasmid carrying AOX1 promoter (i), cloning of plasmid carrying gene (ii), transfer of recombinant plasmid carrying AOX1 promoter and gene to expression host (iii) and providing both induction of AOX1 promoter and expression of associated heterologous gene, using a nitric oxide donor (iv). The method enables expression of various genes of various microorganisms, plants, animals and human, and thus extracellular and/or intracellular production of various recombinant proteins.

### TECHNICAL FIELD

Invention relates to a method providing expression of various genes of various micro-organisms, plants, animals and human and thus production of various recombinant proteins.

### PRESENT STATE OF THE ART

Recently inducible expression systems highly attract attention of researchers and multiple recombinant protein can be produced by such systems. In this context, the methylotrophic yeast Pichia pastoris, which can use methanol as carbon and energy source, is one of the most important expression systems used for both intra- and extracellular recombinant protein production. During last 30 years more than 5000 different proteins are produced by P. pastoris. Mostly, promoter (PAOX1) of alcohol oxidase 1 (AOX1) gene encoding alcohol oxidase (AOX) enzyme play an essential role in production of recombinant proteins at high levels with this popular expression system. The promoter is controlled strongly and strictly by methanol induction. In this line, expression of human, plant, animal and microorganism genes in P. pastoris is possible.

On the other hand, the promoter is repressed by glucose and glycerol. In other words, induction of the AOX1 promoter by methanol enables P. pastoris to produce a high amount of recombinant protein.

Regarding the documents in the related art, patent document numbered US2019309339A1 discloses recombinant protein production process wherein Pichia pastoris yeast is used. The invention states that cultivation is made by both glycerol and methanol.

Another patent document in the related art is the document numbered TR97/01010 and it relates to DNA molecules, recombinant vectors and cell cultures for use in methods for expression of bile salt simulation lipase (BSSL) in Pichia pastoris, a methylotrophic yeast. It discloses that said DNA molecule comprises a human BSSL or a region of a polypeptide that encode a biologically active variant thereof; joined to the 5′-end of said polypeptide coding region, a region coding for a signal peptide capable of directing secretion of said polypeptide from Pichia pastoris cells transformed with said DNA molecule and operably-linked to said coding regions.

However, use of methanol in methanol dependent expression systems has some disadvantages. One of the disadvantages is that methanol is flammable and toxic. As methanol is used in high quantities in recombinant protein production, its storage thereof also causes some technical difficulties. At least 5 mL/L methanol use is needed during recombinant protein production in industrial scale. In addition, high oxygen supply is needed for catabolism and this means increase in production cost as high quantity of heat release is caused. Methanol is not preferred for production of pharmaceuticals or edible products. Hydrogen peroxide (H₂O₂) which is a side product of metabolism causes oxidative stress and thus causes proteolytic degradation of recombinant proteins. In addition, as methanol is a petroleum product, its cost increases in petroleum crisis.

In order to eliminate these advantages caused by use of methanol in recombinant protein production, studies on methanol free Pichia pastoris expression system have increased in the literature.

In one of them, Shirvani et al. (2019) developed a methanol-free expression system for extracellular production of human granulocyte macrophage colony simulator factor (hGM-CSF), which plays an important role in reproduction and differentiation of immune cells and is a pharmaceutic protein. For this purpose, a new expression vector [pEP (α) 101], carrying the pFMD (formate dehydrogenase) promoter regulating the expression of heterologous gene by use of glycerol was designed. Expression of recombinant hGM-CSF in three different culture media is studied. According to results, it was reported that new methanol free pFMD expression system would be a suitable candidate for production of food-grade and therapeutically important recombinant proteins.

Takagi et al. (2019) developed a methanol-free Pichia pastoris expression system via arrangement of a strong methanol-inducible DAS1 (dihydroxyacetone synthase) promoter, using Citrobacter braakii phytase enzyme production as a model. For that purpose, constitutive expression of KpTRM1 (positive transcription regulator of genes associated with methanol use in Pichia pastoris) was performed using DAS1 promoter not AOX1 promoter and it was shown that this promoter enabled to yeast cells to produce phytase without adding methanol to medium.

Another example in the literature is the study of Wand et al. (2017) where to obtain methanol free P. pastoris expression system, functions of trans-acting factors of AOX1 promoter are tested and these factors are arranged combinatorially. In the study three transcription receptors (Mig1, Mig2 and Nrg1) are defined and deleted and a transcription activator (Mit1) is over expressed, thus a methanol-free P_{AOX1} start-up strain carrying GFP gene in the downstream region of AOX1 promoter was constructed.

When compared to wild strain where production is performed by use of methanol, this strain produced 77% more GFP in glycerol, AOX promoter repressor. Then it became possible to produce protein without use of methanol in the P. pastoris strain capable to produce insulin precursor (IP) protein. It is reported that the prepared mutant and used biological processes in the study offer a safe and efficient alternative to conventional glycerol-repressed/methanol-induced P_{AOX1} system.

In another study Shen et al. (2016) focused on constructing new methanol-free expression system targeting kinases playing role in activation or inhibition of AOX1 promoter by different carbon sources. For this purpose, two kinase mutants (Δgut1 and Δdak) were constructed, and it was seen that they had strong alcohol oxidase activity without methanol. In conclusion, they constructed two different methanol-free expression systems: Δgut1-HpGCY1-glycerol (P_{AOX1} promoter inducible by glycerol) and Δdak-DHA (P_{OAX1} promoter inducible by dehydroxiasetone). When two mutant systems were compared, Δdak-DHA system was found to better. In addition, it was reported that methanol-free Δdak-DHA expression system was better than constitutive GAP promoter, and the system could reach 50-60% of the conventional methanol-induced (wild type) system.

In these studies, available in the literature and achieved without use of methanol, it was aimed to investigate new promoters alternative to AOX promoter, to inhibit and/or activate current transcription factors and to obtain mutant strains capable to express methanol-independent. Such types of genetic manipulations cause long and dedicated processes in recombinant protein production with P. pastoris as well as high costs. Also, the studies conducted indicate that methanol free expression systems intended to be developed in generally provide lower costs in comparison to methanol-dependent ones. This implies that the systems intended to be developed are not advantageous in respect to product efficiency. In short, even if methanol free expression systems offered as solution eliminate the use of methanol, the decrease in efficiency emerges as a technical problem.

When studies in the related art are examined it is seen that innovative recombinant protein production methods providing low cost and high efficient and methanol-independent production without need for genetic intervention are still needed today.

### BRIEF DESCRIPTION OF THE INVENTION

The present invention relates to a recombinant protein production method meeting the needs mentioned above, eliminating all disadvantages, and providing some additional advantages.

Primary purpose of the invention is to develop a method that enables expression of gene inserted into downstream of the P_{AOX1} and production of recombinant protein by this yeast without use of methanol.

Another purpose of the invention is to develop a recombinant protein production method providing higher yields in comparison to methanol free systems in the related art despite methanol independency.

A further purpose of the invention is to develop a recombinant protein production method wherein a compound which is a nitric oxide donor and not displaying toxic property unlike methanol is used for induction of AOX1 promoter instead of methanol.

Another purpose of the invention is to develop a recombinant protein production method reducing amount of inducer and thus cost thereof.

Another purpose of the invention is to develop a recombinant protein production method eliminating problem of storing inducer and burning danger thereof.

A further purpose of the invention is to develop a recombinant protein production method not requiring any genetic intervention onto promoter region and transcription factors for heterologous gene expression.

Another purpose of the invention is to develop a recombinant protein production method reducing workload and cost in industrial scale.

In order to achieve above mentioned purposes, the present invention is a method for extracellular and intracellular recombinant protein production comprising process steps of
i. insertion of gene to be expressed into the plasmid carrying AOX1 promoter
ii. cloning of plasmid carrying gene
iii. transfer of recombinant plasmid carrying AOX1 promoter and gene to expression host
iv. providing both induction of AOX1 promoter and expression of heterologous gene, using a nitric oxide donor.

The structural and characteristic features and all advantages of the invention will be understood better in the figure given below and the detailed description by reference to the figure. Therefore, the assessment should be made taking into account the detailed description.

### BRIEF DESCRIPTION OF THE DRAWINGS

FIG. 1 is efficiency analysis graphic of heterologous gene expression levels giving comparison of method of invention to control groups of different applications.

### DESCRIPTION OF REFERENCES

A. Growth culture containing methanol in 0.5% in volume

B. Growth culture containing glucose in 1% and methanol in 0.5% in volume

C. Growth culture containing glucose in 1% and 800 nM sodium nitroprusside

D. Growth culture containing glycerol in 1% and methanol in 0.5% in volume

E. Growth culture containing glycerol in 1% and 800 nM sodium nitroprusside

F. Growth culture containing sorbitol in 1% and methanol in 0.5% in volume

G. Growth culture containing sorbitol in 1% and 800 nM sodium nitropusside

### DETAILED DESCRIPTION OF THE INVENTION

In this detailed description, a method for recombinant protein production being subject of this invention and the preferred applications have been disclosed for the purpose of better understanding of the subject and described in a manner not causing any restrictive effect.

Invention is a method for extracellular and intracellular recombinant protein production comprising process steps of
i. Insertion of gene to be expressed into the plasmid carrying AOX1 promoter
ii. cloning of plasmid carrying gene
iii. transfer of recombinant plasmid carrying AOX1 promoter and gene to expression host
iv. providing both induction of AOX1 promoter and expression of associated heterologous gene, using a nitric oxide donor.

‘gene” mentioned in process step number (i) is a nucleotide sequence of protein intended to be produced recombinantly and is obtained from a living being selected from a group comprising human, plant, animal and microorganisms.

In an application of the invention said gene is prepromelittin protein gene of honeybee (Apis mellifera). In another application of the invention, said gene is human PINX1 (hPINX1) protein gene.

According to a preferred application of the invention said plasmid is selected from a group comprising Pichia pastoris plasmids carrying AOX promoters such as pPICZα (A, B and C) and pPICZ (A, B and C).

In an application of the invention, in processes step no (ii) bacteria cells are used for cloning of heterologous gene inserted into plasmid. In a preferred application of the invention said bacterial cells are E. coli cells, most preferably E. coli TOP10 strain.

In another application of the invention, said expression host in process step no (iii) is selected from a group comprising yeasts (Pichia pastoris, Saccharomyces cerevisiae, Hansenula polymorpha, Yarrowia lipolytica and Kluyveromyces lactis), bacteria (Escherichia coli, Bacillus subtilis and Lactococcus lactis) and plants (Arabidopsis sp. and Nicotiana tabacum).

In a preferred application of the invention the expression host mentioned in process step no (iii) is the recombinant P. pastoris cells that carry the protein gene transferred to the plasmid in its genome and perform production of this protein thereof extracellularly.

In a preferred application of the invention, nitric oxide donor mentioned in process step no (iv) is used as inducer.

Nitric oxide (NO) is a water and fat soluble gas molecule produced endogenously in bacteria, yeast, plants and humans. The molecule has different physiological functions in said organisms. For instance, it is reported that in higher plants endogenous NO plays a significant role in plant growth and development, seed germination, primary and lateral root growth, flowering, fruit ripening, senescence, respiration and stomatal closure, and adaptive responses against biotic-abiotic stresses. NO produced as endogenous in human body functions as a signal molecule and plays role in the regulation of immunological responses, blood flow, secretion, oxygen perception and respiratory energy reproduction.

Due to mentioned beneficial properties of NO, the application of exogenous NO to biological systems for biotechnological and medicinal purposes is seen as an important approach. In this line, NO donors providing exogenous NO are applied to biological systems. Released exogenous NO mimics response of endogenous NO or completes its deficiency.

In a preferred application of the invention, NO donor is selected from a group comprising sodium nitroprusside, glyceryl trinitrate, isoamyl nitrite, isosorbide dinitrate, isosorbide mononitrate, s-nitrosoglutathione, s-nitroso-n-acetylcysteine, s-nitroso-N-acetylpenicillamine, diethylamine NONOate and spermine NONOate. According to a preferred application, sodium nitroprusside (SNP) is used as an inducer.

In a preferred application of the invention, in process step of (iv) at least a carbon source selected from a group comprising sorbitol, mannitol, trehalose and glycerol is used. In addition to carbon sources such as sorbitol, mannitol and trehalose not natural AOX promoter repressors, glycerol can also be used as carbon source since SNP induction eliminates repressing property of glycerol on promoter.

SNP used in the most preferred application of the invention is used not only plant but also used in human studies as exogenous NO donor. NO donor (SNP) applied as exogenous is evidenced to reduce salt stress, heavy metal toxicity, chilling stress and herbicide damage in plants by SNP. In literature it is reported that in human exogenously-applied NO donor (SNP) is used as a drug in treatment of coronary heart disease (as vasodilator), regulation of basophil and mast cell activation, sperm motility, and synthesis of type I collagen and heat shock protein. These results all show that NO and NO donor-SNP do not have toxic effect on humans, on contrary, they can be used as drug.

In the current invention NO donors, preferably SNP increases recombinant protein expression surprisingly when used as inducer on AOX1 promoter.

SNP is not toxic, even small amounts thereof is functional in industrial scale, does not cause storing problem and is not flammable, and because of these properties its preferability is increased. In addition, use of SNP eliminates problem of breaking of sodium alginate beads and enables sodium alginate-immobilized P. pastoris cells to produce extracellular recombinant protein in continuous cultures.

On the other hand, with use of SNP as inducer, there is no need for any genetic intervention (regulation, mutation, over expression or knock-down etc) on promoter region and transcription factors for expression of heterologous gene. Therefore, essential contribution to reduce workload and cost is provided. The fact that SNP applied as a NO donor is cheap and performs heterologous gene induction at even very low concentrations also reveals that the invention can be applied at low cost on an industrial scale.

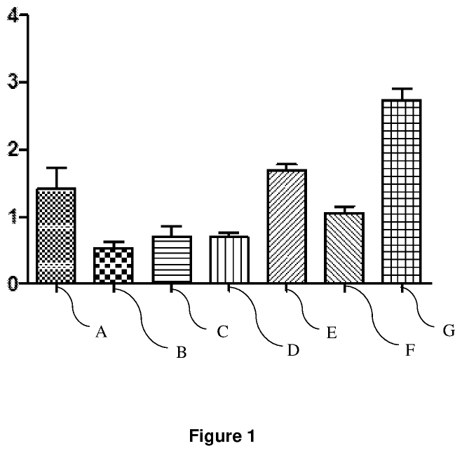

The most importantly, with use of SNP, much higher protein production can be achieved in comparison to methanol induction. According to heterologue gene expression analysis conducted, SNP induction showed similar results in the presence of glycerol compared to only methanol induction (no significant difference, p>0.05), in the presence of sorbitol a higher gene expression level was observed with SNP induction (FIG. 1). Therefore, with the method disclosed under the invention, workload and cost are reduced as well as recombinant protein quantity is considerably increased.

In a study conducted under scope of the invention, to produce recombinant protein, recombinant P. pastoris cells (constructed in our laboratory) carrying “prepromelittin protein” gene of Apis mellifera (honey bee) in its genome and producing extracellularly this protein were used. With the method of the invention, it was observed that extracellular protein production was achieved in groups, where SNP was used as inducer (at various concentrations of 200, 400, 600 and 800 nanomoles) for induction of promoter region and sorbitol was as carbon source.

In another study performed under scope of the invention, human PINX1 (hPINX1) protein under AOX1 promoter control by adding SNP in 800 nM concentration was produced intracellularly with P. pastoris. According to Western blot analysis result of this study, it was seen that SNP induced hPINX1 protein production.

As a result, method of the invention enables the conversion of human, plant, animal and microorganism genes to extracellular and intracellular recombinant proteins (insulin, growth factors etc.).

1. A method for extracellular and intracellular recombinant protein production comprising the process steps of:
i. insertion of a gene to be expressed into a plasmid carrying AOX1 promoter;
ii. cloning of plasmid carrying gene;
iii. transfer of recombinant plasmid carrying AOX1 promoter and gene to an expression host; and
iv. providing both induction of AOX1 promoter and expression of associated heterologous gene, using a nitric oxide donor.

2. The method according to claim 1, wherein the gene mentioned in process step no (i) is a nucleotide sequence of protein intended to be produced recombinantly and is obtained from a living being selected from a group comprising human, plant, animal and microorganisms.

3. The method according to claim 2, wherein the gene mentioned in process step (i) being a prepromelittin protein gene of honey bee.

4. The method according to claim 2, wherein the gene mentioned in process step (i) being a PINX1 protein gene.

5. The method according to claim 1, wherein the plasmid mentioned in process step (i) being selected from a group comprising pPICZα (A, B and C) plasmid, pPICZ (A, B and C) plasmid and other P. pastoris plasmids carrying AOX promoter.

6. The method according to claim 1, wherein the bacteria cells are used for cloning of heterologous gene inserted into plasmid in process step no(ii).

7. The method according to claim 6, wherein said bacteria cells are E. coli cells.

8. The method according to claim 7, wherein said E. coli cells are E. coli TOP10 strain cells.

9. The method according to claim 1, wherein the expression host mentioned in process step no (iii) being selected from a group comprising yeasts (Pichia pastoris, Saccharomyces cerevisiae, Hansenula polymorpha, Yarrowia hpolytica and Kluyveromyces lactis), bacteria (Esherichia coli, Bacillus subtilis, and Lactococcus lactis) and plants (Arabidopsis sp. and Nicotiana tabacum).

10. The method according to claim 9, wherein the expression host mentioned in process step no (iii) is the recombinant P. pastoris cells that carry the protein gene transferred to plasmid and perform production of this protein thereof extracellularly.

11. The method according to claim 1, wherein NO donor used as inducer in process step no (vi) being selected from a group comprising sodium nitroprusside, glyceryl trinitrate, isoamyl nitrite, isosorbide dinitrate, isosorbide mononitrate, s-nitrosoglutathione, s-nitroso-n-acetylcysteine, s-nitroso-N-acetylpenicillamine, diethylamine NONOate and spermine NONOate.

12. The method according to claim 11, wherein NO donor is sodium nitroprusside.

13. The method according to claim 11, wherein at least a carbon source selected from a group comprising sorbitol, mannitol, trehalose and glycerol being used additionally in the process step no (vi).
